(19) United States
(12) Patent Application Publication
(10) Pub. No.: US 2002/0049320 A1
(43) Pub. Date: Apr. 25, 2002

(54) 6-amino-2,4-dioxo-3,4-dihydro-1,3,5-triazine derivatives and methods for the solid phase synthesis thereof

(75) Inventors: Ariamala Gopalsamy, Mahwah, NY (US); Hui Y. Yang, New City, NY (US)

Correspondence Address: Egon E. Berg, American Home Products Corporation, Patent Law Department, Five Giralda Farms, Madison, NJ, 07940, US

(73) Assignee: American Cyanamid Company, Madison, NJ

(21) Appl. No.: 09/825,979

(22) Filed: Apr. 4, 2001

Related U.S. Application Data
(60) Provisional application No. 60/199,952, filed on Apr. 27, 2000.

(51) Int. Cl. C 07D 4 3/02; C07D 251/42

(52) U.S. Cl. 544/194; 544/212

(57) ABSTRACT

The present invention relates to substituted 6-amino-2,4-dioxo-3,4-dihydro-1,3,5-triazine compounds of formula (I)

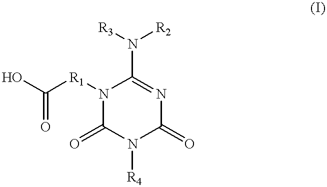

having pharmacological activity, to solid phase synthesis methods for their preparation, to combinatorial libraries thereof, utilizing libraries of the compounds for drug discovery, and to pharmaceutical compositions thereof.

This application claims the benefit of U.S. Provisional Application No. 60/199,952, filed Apr. 27, 2000.

### FIELD OF THE INVENTION

The present invention relates to 6-amino-2,4-dioxo-3,4-dihydro-1,3,5-triazine derivatives, to processes for their preparation, to combinatorial and solid phase methods for preparing libraries of the compounds, to utilizing libraries of the compounds for drug discovery, and to pharmaceutical compositions thereof.

### BACKGROUND OF THE INVENTION

The solid phase synthesis of non-peptidic small organic molecules is a rapidly evolving area of research with applications in the preparation of combinatorial libraries. While the solid phase synthesis of peptides is well established, the solid phase synthesis of non-peptidic small organic molecules is still evolving (Hermkens, P. H. H.; Ottenheijm, H. C. J.; Rees, D. Tetrahedron 1996, 52, 4527-4554). In particular, methods for the solid phase synthesis of heterocyclic ring systems of importance to drug discovery is an active area of research.

Triazine derivatives are important class of molecules with physiological significance and pharmaceutical and agrochemical utility. Substituted 1,3,5-triazine-2,4-diones are a useful class of compounds. Brown, et al. (EP 5911, 1979) describe 6-acylaminotetrahydro-1,3,5-trazine-2,4-dione derivatives to possess analgesic properties. In addition 1,3,5-triazine-2,4-dione derivatives possess herbicidal properties (GB 1464248).

Combinatorial chemistry is becoming an important tool for drug discovery and lead optimization (Borman, S. Chemical and Engineering News 1997, 75 (8), 43-62). A combinatorial synthesis requires that at least two components of the product molecules be independently variable, so that all of the combinations of these components can be prepared. Thus, to prepare a combinatorial library of 1,3,5-triazine-2,4-dione derivatives with a high degree of potential diversity and wide utility for drug discovery using solid phase techniques, it is important to identify a synthesis in which all the components can be independently varied. The solution phase synthesis of 1,3,5-triazine-2,4-diones by reaction of ureas with chlorocarbonylisocyanate is known (Hagemann, H. Angew. Chem. Int. Ed. Engl.. 1977, 16, 743-750). For a solid phase combinatorial synthesis it is necessary to modify this syntheses to allow for the independent introduction of variables and to adapt the solution phase synthesis to a solid supported synthesis. The solid phase 1,3,5-triazine-2,4-dione synthesis of this invention is achieved by using a variety of amino acids as starting material which can be attached to a solid support through the carboxylic acid group.

Multiple compounds can be prepared simultaneously by the solid phase process. The simultaneous solid phase synthesis of a library of trisubstituted 1,3,5-triazine-2,4-diones of the present invention is not known. The preparation of libraries of compounds of the present invention is useful because it provides rapid structural variation and structure-activity information.

The libraries of substituted 1,3,5-triazine-2,4-diones synthesized according to the present invention are useful for drug discovery.

### BRIEF DESCRIPTION OF THE INVENTION

Accordingly, the present invention provides compounds represented by the formula (I):

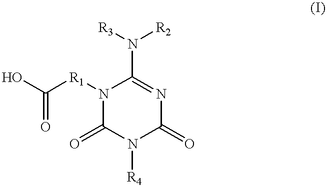

wherein:

R¹ is

an amino acid side chain or phenyl optionally substituted with one or two substituents selected from halogen, straight chain alkyl of 1 to 6 carbon atoms, branched chain alkyl of 2 to 6 carbon atoms, or alkoxy of 1 to 6 carbon atoms;

R² and R³ are independently

hydrogen, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms, phenylalkyl of 7 to 12 carbon atoms, phenyl or acyl; or R² and R³ taken together with the atom to which they are attached, form a 5 or 6 membered heterocyclic ring having one to three heteroatoms selected from O, N and S;

R⁴ is

hydrogen, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms, phenylalkyl of 7 to 12 carbon atoms wherein the phenyl group may be optionally substituted with 1 or 2 substituents selected from halogen, trifluoromethyl, straight chain or branched alkoxy of 2 to 7 carbon atoms, phenylalkoxy of 7 to 12 carbon atoms, nitro, methylenedioxy, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms or

two substituents on adjacent carbons taken together with the phenyl ring form naphthyl optionally substituted with 1 or 2 substituents selected from halogen, straight chain or branched alkoxy of 2 to 7 carbon atoms, nitro, methylenedioxy, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms;

and all crystalline forms and the pharmaceutically acceptable salts thereof, the enantiomers thereof, the racemic mixtures thereof, and the diastereomeric mixtures.

The present invention further provides a combinatorial library of compounds of Formula I.

In some preferred embodiments of the present invention, R¹ is phenyl optionally substituted with halogen. In other preferred embodiments of the present invention R² and R³ taken together form piperidine. Alternatively, R² is alkyl and R³ is alkyl or phenylalkyl. R⁴ is preferably phenylalkyl.

In accordance with other preferred embodiments of the present invention, R1 is phenyl, R2 and R3 together with the atom to which they are attached, form a heterocyclic ring, and R4 is phenylalkyl.

Preferred compounds of the present invention are

4-(3-(4-bromobenzyl)-2,4-dioxo-6-piperidin-1-yl-3,4-dihydro-1,3,5-triazin-1(2H)-yl)benzoic acid;

4-(6-[benzyl(ethyl)amino]-3-(4-bromobenzyl)-2,4-dioxo-3,4-dihydro-1,3,5-triazin-1(2H)-yl)benzoic acid;

4-(3-(4-bromobenzyl)-6-(dipropylamino)-2,4-dioxo-3,4-dihydro-1,3,5-triazin-1(2H)-yl)benzoic acid;

3-(3-(4-bromobenzyl)-2,4-dioxo-6-piperidin-1-yl-3,4-dihydro-1,3,5-triazin-1(2H)-yl)benzoic acid;

4-(3-(4-bromobenzyl)-2,4-dioxo-6-piperidin-1-yl-3,4-dihydro-1,3,5-triazin-1(2H)-yl)-2-chlorobenzoic acid;

5-(3-(4-bromobenzyl)-2,4-dioxo-6-piperidin-1-yl-3,4-dihydro-1,3,5-triazin-1(2H)-yl)-2-chlorobenzoic acid;

4-(2,4-dioxo-6-piperidin-1-yl-3-[4-(trifluoromethyl)benzyl]-3,4-dihydro-1,3,5-triazin-1(2H)-yl)benzoic acid;

4-(3-[4-(benzyloxy)benzyl]-2,4-dioxo-6-piperidin-1-yl-3,4-dihydro-1,3,5-triazin-1(2H)-yl)benzoic acid;

4-(3-(3,4-dimethylbenzyl)-2,4-dioxo-6-piperidin-1-yl-3,4-dihydro-1,3,5-triazin-1(2H)-yl)benzoic acid;

4-(3-([1,1′-biphenyl]-2-ylmethyl)-2,4-dioxo-6-piperidin-1-yl-3,4-dihydro-1,3,5-triazin-1(2H)-yl)benzoic acid;

4-(6-[benzyl(ethyl)amino]-2,4-dioxo-3-[4-(trifluoromethyl)benzyl]-3,4-dihydro-1,3,5-triazin-1(2H)-yl)benzoic acid;

4-(6-[benzyl(ethyl)amino]-3-[4-(benzyloxy)benzyl]-2,4-dioxo-3,4-dihydro-1,3,5-triazin-1(2H)-yl)benzoic acid;

4-(6-[benzyl(ethyl)amino]-3-(3,4-dimethylbenzyl)-2,4-dioxo-3,4-dihydro-1,3,5-triazin-1(2H)-yl)benzoic acid;

4-(6-[benzyl(ethyl)amino]-3-([1,1′-biphenyl]-2-ylmethyl)-2,4-dioxo-3,4-dihydro-1,3,5-triazin-1(2H)-yl)benzoic acid;

4-(6-(dipropylamino)-2,4-dioxo-3-[4-(trifluoromethyl)benzyl]-3,4-dihydro-1,3,5-triazin-1(2H)-yl)benzoic acid;

4-(3-[4-(benzyloxy)benzyl]-6-(dipropylamino)-2,4-dioxo-3,4-dihydro-1,3,5-triazin-1(2H)-yl)benzoic acid;

4-(3-(3,4-dimethylbenzyl)-6-(dipropylamino)-2,4-dioxo-3,4-dihydro-1,3,5-triazin-1(2H)-yl)benzoic acid;

4-(3-([1,1′-biphenyl]-2-ylmethyl)-6-(dipropylamino)-2,4-dioxo-3,4-dihydro-1,3,5-triazin-1(2H)-yl)benzoic acid;

3-(2,4-dioxo-6-piperidin-1-yl-3-[4-(trifluoromethyl)benzyl]-3,4-dihydro-1,3,5-triazin-1(2H)-yl)benzoic acid;

3-(3-[4-(benzyloxy)benzyl]-2,4-dioxo-6-piperidin-1-yl-3,4-dihydro-1,3,5-triazin-1(2H)-yl)benzoic acid;

3-(3-(3,4-dimethylbenzyl)-2,4-dioxo-6-piperidin-1-yl-3,4-dihydro-1,3,5-triazin-1(2H)-yl)benzoic acid;

3-(3-([1,1′-biphenyl]-2-ylmethyl)-2,4-dioxo-6-piperidin-1-yl-3,4-dihydro-1,3,5-triazin-1(2H)-yl)benzoic acid;

3-(6-[benzyl(ethyl)amino]-3-(4-bromobenzyl)-2,4-dioxo-3,4-dihydro-1,3,5-triazin-1(2H)-yl)benzoic acid;

3-(6-[benzyl(ethyl)amino]-2,4-dioxo-3-[4-(trifluoromethyl)benzyl]-3,4-dihydro-1,3,5-triazin-1(2H)-yl)benzoic acid;

3-(6-[benzyl(ethyl)amino]-3-[4-(benzyloxy)benzyl]-2,4-dioxo-3,4-dihydro-1,3,5-triazin-1(2H)-yl)benzoic acid;

3-(6-[benzyl(ethyl)amino]-3-(3,4-dimethylbenzyl)-2,4-dioxo-3,4-dihydro-1,3,5-triazin-1(2H)-yl)benzoic acid;

3-(6-[benzyl(ethyl)amino]-3-([1,1′-biphenyl]-2-ylmethyl)-2,4-dioxo-3,4-dihydro-1,3,5-triazin-1(2H)-yl)benzoic acid;

3-(3-(4-bromobenzyl)-6-(dipropylamino)-2,4-dioxo-3,4-dihydro-1,3,5-triazin-1(2H)-yl)benzoic acid;

3-(6-(dipropylamino)-2,4-dioxo-3-[4-(trifluoromethyl)benzyl]-3,4-dihydro-1,3,5-triazin-1(2H)-yl)benzoic acid;

3-(3-[4-(benzyloxy)benzyl]-6-(dipropylamino)-2,4-dioxo-3,4-dihydro-1,3,5-triazin-1(2H)-yl)benzoic acid;

3-(3-(3,4-dimethylbenzyl)-6-(dipropylamino)-2,4-dioxo-3,4-dihydro-1,3,5-triazin-1(2H)-yl)benzoic acid;

3-(3-([1,1′-biphenyl]-2-ylmethyl)-6-(dipropylamino)-2,4-dioxo-3,4-dihydro-1,3,5-triazin-1(2H)-yl)benzoic acid;

2-chloro-4-(2,4-dioxo-6-piperidin-1-yl-3-[4-(trifluoromethyl)benzyl]-3,4-dihydro-1,3,5-triazin-1(2H)-yl)benzoic acid;

4-(3-[4-(benzyloxy)benzyl]-2,4-dioxo-6-piperidin-1-yl-3,4-dihydro-1,3,5-triazin-1(2H)-yl)-2-chlorobenzoic acid;

2-chloro-4-(3-(3,4-dimethylbenzyl)-2,4-dioxo-6-piperidin-1-yl-3,4-dihydro-1,3,5-triazin-1(2H)-yl)benzoic acid;

4-(3-([1,1′-biphenyl]-2-ylmethyl)-2,4-dioxo-6-piperidin-1-yl-3,4-dihydro-1,3,5-triazin-1(2H)-yl)-2-chlorobenzoic acid;

4-(6-[benzyl(ethyl)amino]-3-(4-bromobenzyl)-2,4-dioxo-3,4-dihydro-1,3,5-triazin-1(2H)-yl)-2-chlorobenzoic acid;

4-(6-[benzyl(ethyl)amino]-2,4-dioxo-3-[4-(trifluoromethyl)benzyl]-3,4-dihydro-1,3,5-triazin-1(2H)-yl)-2-chlorobenzoic acid;

4-(6-[benzyl(ethyl)amino]-3-[4-(benzyloxy)benzyl]-2,4-dioxo-3,4-dihydro-1,3,5-triazin-1(2H)-yl)-2-chlorobenzoic acid;

4-(6-[benzyl(ethyl)amino]-3-(3,4-dimethylbenzyl)-2,4-dioxo-3,4-dihydro-1,3,5-triazin-1(2H)-yl)-2-chlorobenzoic acid;

4-(6-[benzyl(ethyl)amino]-3-([1,1′-biphenyl]-2-ylmethyl)-2,4-dioxo-3,4-dihydro-1,3,5-triazin-1(2H)-yl)-2-chlorobenzoic acid;

4-(3-(4-bromobenzyl)-6-(dipropylamino)-2,4-dioxo-3,4-dihydro-1,3,5-triazin-1(2H)-yl)-2-chlorobenzoic acid;

2-chloro-4-(6-(dipropylamino)-2,4-dioxo-3-[4-(trifluoromethyl)benzyl]-3,4-dihydro-1,3,5-triazin-1(2H)-yl)benzoic acid;

4-(3-[4-(benzyloxy)benzyl]-6-(dipropylamino)-2,4-dioxo-3,4-dihydro-1,3,5-triazin-1(2H)-yl)-2-chlorobenzoic acid;

2-chloro-4-(3-(3,4-dimethylbenzyl)-6-(dipropylamino)-2,4-dioxo-3,4-dihydro-1,3,5-triazin-1(2H)-yl)benzoic acid;

4-(3-([1,1′-biphenyl]-2-ylmethyl)-6-(dipropylamino)-2,4-dioxo-3,4-dihydro-1,3,5-triazin-1(2H)-yl)-2-chlorobenzoic acid;

2-chloro-5-(2,4-dioxo-6-piperidin-1-yl-3-[4-(trifluoromethyl)benzyl]-3,4-dihydro-1,3,5-triazin-1(2H)-yl)benzoic acid;

5-(3-[4-(benzyloxy)benzyl]-2,4-dioxo-6-piperidin-1-yl-3,4-dihydro-1,3,5-triazin-1(2H)-yl)-2-chlorobenzoic acid;

2-chloro-5-(3-(3,4-dimethylbenzyl)-2,4-dioxo-6-piperidin-1-yl-3,4-dihydro-1,3,5-triazin-1(2H)-yl)benzoic acid;

5-(3-([1,1′-biphenyl]-2-ylmethyl)-2,4-dioxo-6-piperidin-1-yl-3,4-dihydro-1,3,5-triazin-1(2H)-yl)-2-chlorobenzoic acid;

5-(6-[benzyl(ethyl)amino]-3-(4-bromobenzyl)-2,4-dioxo-3,4-dihydro-1,3,5-triazin-1(2H)-yl)-2-chlorobenzoic acid;

5-(6-[benzyl(ethyl)amino]-2,4-dioxo-3-[4-(trifluoromethyl)benzyl]-3,4-dihydro-1,3,5-triazin-1(2H)-yl)-2-chlorobenzoic acid;

5-(6-[benzyl(ethyl)amino]-3-[4-(benzyloxy)benzyl]-2,4-dioxo-3,4-dihydro-1,3,5-triazin-1(2H)-yl)-2-chlorobenzoic acid;

5-(6-[benzyl(ethyl)amino]-3-(3,4-dimethylbenzyl)-2,4-dioxo-3,4-dihydro-1,3,5-triazin-1(2H)-yl)-2-chlorobenzoic acid;

5-(6-[benzyl(ethyl)amino]-3-([1,1′-biphenyl]-2-ylmethyl)-2,4-dioxo-3,4-dihydro-1,3,5-triazin-1(2H)-yl)-2-chlorobenzoic acid;

5-(3-(4-bromobenzyl)-6-(dipropylamino)-2,4-dioxo-3,4-dihydro-1,3,5-triazin-1(2H)-yl)-2-chlorobenzoic acid;

2-chloro-5-(6-(dipropylamino)-2,4-dioxo-3-[4-(trifluoromethyl)benzyl]-3,4-dihydro-1,3,5-triazin-1(2H)-yl)benzoic acid;

5-(3-[4-(benzyloxy)benzyl]-6-(dipropylamino)-2,4-dioxo-3,4-dihydro-1,3,5-triazin-1(2H)-yl)-2-chlorobenzoic acid;

2-chloro-5-(3-(3,4-dimethylbenzyl)-6-(dipropylamino)-2,4-dioxo-3,4-dihydro-1,3,5-triazin-1(2H)-yl)benzoic acid; and

5-(3-([1,1′-biphenyl]-2-ylmethyl)-6-(dipropylamino)-2,4-dioxo-3,4-dihydro-1,3,5-triazin-1(2H)-yl)-2-chlorobenzoic acid; and pharmaceutical salts thereof.

It is understood that the definition of the compounds of formula (I), when R¹, R², R³, and R⁴ contain asymmetric carbons, encompasses all possible stereoisomers and mixtures thereof. In particular, it encompasses racemic modifications and any optical isomers. Optical isomers may be obtained in pure form by standard separation techniques. The pharmaceutically acceptable salts are those derived from such organic and inorganic acids as: lactic, citric, acetic, tartaric, succinic, maleic, malonic, hydrochloric, hydrobromic, phosphoric, nitric, sulfuric, methanesulfonic, and similarly known acceptable acids. Carboxylic acid salts of the compounds of this invention may be formed with bases such as alkali metals (Na, K, Li) or the alkaline earth metals (Ca or Mg).

The term “amino acid side chain”, as used herein, is meant to refer to the side chain of amino acids including, but not limited to naturally occuring amino acids such as glycine, alanine, valine, leucine, isoleucine, lysine, arginine, histidine, phenylalanine, tyrosine, tryptophan, aspartate, glutamate, asparaine, glutamine, cysteine and methionine. The side chains of naturally occuring amino acid side chains are well known in the art. One of skill in the art will also appreciate that amino acid also include β-, γ-, δ-, and ω-amino acids.

Halogen means chlorine, bromine, fluorine and iodine.

Heterocyclic rings are 5 to 6 membered monocyclic rings having 1 to 3 heteroatoms selected from nitrogen, oxygen and sulfur, including, but not limited to piperidine, piperazine, imidazolidine, morpholine, pyrrolidine and pyrazolidine.

In further embodiments of the present invention, compounds of Formula I may be prepared by solid phase synthesis comprising the steps of:

a) attaching a Fmoc protected amino acid of the formula

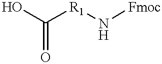

to a solid support to produce a compound of formula (1)

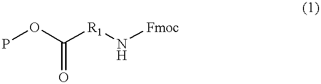

wherein R¹ is as defined above and P is a solid support, preferably a polystyrene resin crosslinked with divinylbenzene and functionalized with a linker such as a hydroxymethylphenoxy group and more preferably Wang's resin as described below;

b) Deprotecting the said compound of formula (1) with piperidine to produce a compound of formula (2)

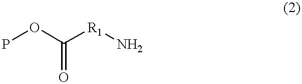

wherein R¹ and P are as defined above;

c) reacting said compound of formula (2) with Fmoc-isothiocyanate to produce a compound of formula (3)

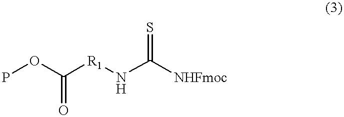

wherein R¹ and P are as defined above;

d) deprotecting the said compound of formula (3) with piperidine to produce a compound of formula (4)

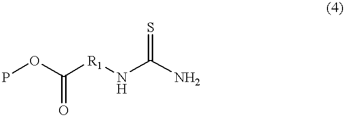

wherein R¹ and P are as defined above;

e) reacting said compound of formula (4) with methyl iodide to produce a compound of formula (5)

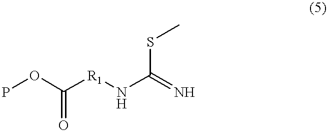

wherein R¹ and P are as defined above;

f) reacting said compound of formula (5) with amines of formula (6) to produce a compound of formula (7)

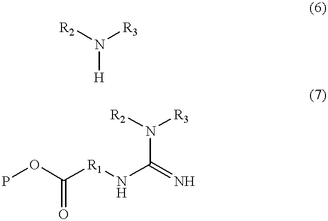

wherein R¹, R², R³ and P are as defined above:

g) reacting compound of formula (7) with chlorocarbonylisocyanate to produce a compound of formula (8)

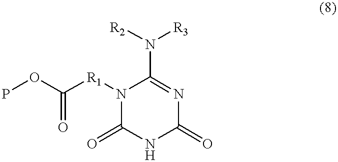

wherein R¹, R², R³ and P are as defined above;

h) reacting said compound of formula (8) with alcohols under mitsunobu condition (Thomas, A. R.; Chapmann, K. T., Tetrahedron Letters, 1995, 36(22) 3789-92) to produce a compound of formula (9)

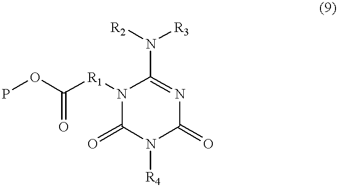

wherein R¹, R², R³ and R⁴ are as defined above; and

i) reacting said compound of formula (9) with a cleaving reagent such as trifluoroacetic acid to produce a compound of formula (I)

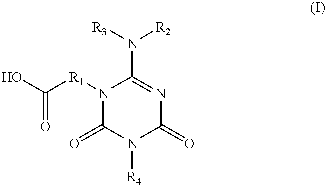

wherein R¹, R², R³ and R⁴ are as defined above.

Libraries of the present invention may be attached to solid supports or cleaved from the support. Solid support include insoluble substrate that has been appropriately derivatized such that a chemical module can be attached to the surface of the substrate through standard chemical methods. Solid supports include, but are not limited to beads and particles.

Compounds of Formula I may be prepared according to the general process outlined below in Scheme I.

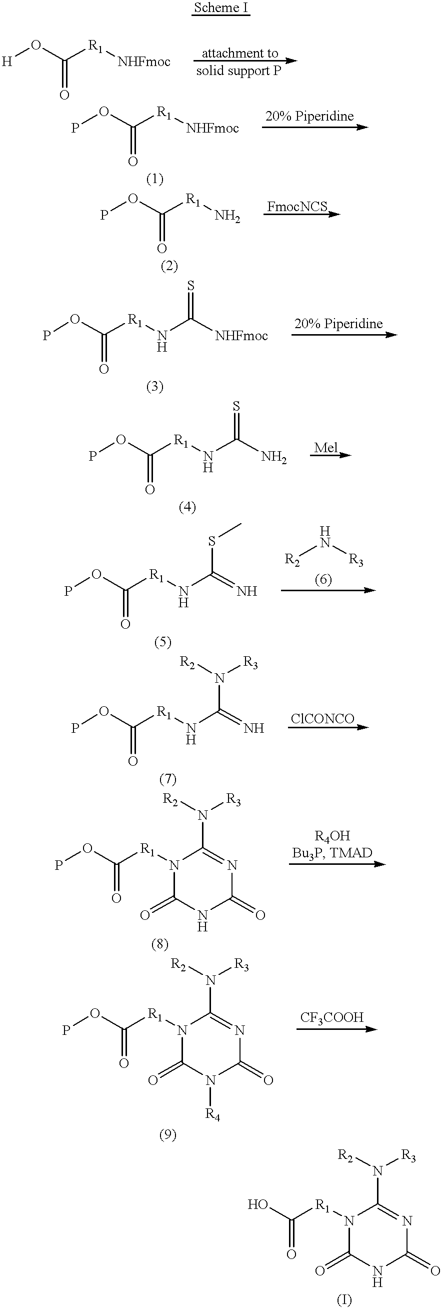

Thus, a Fmoc protected amino acid is attached to the preferred solid support P, a resin of polystyrene crosslinked with divinylbenzene and with a linker such as 4-hydroxymethylphenoxy, most preferably Wang's resin (Wang S.; J. Am. Chem. Soc. 1973, 95, 1328-1333) in the presence of a coupling reagent such as diisopropylcarbodiimide to produce a compound of formula (1). A compound of formula (1) is deprotected using 20% piperidine in DMF to produce a free amine of formula (2). Amine (2) is reacted with fluorenylmethyloxycarbonyl isothiocyanate (Kearney et al. J. Org. Chem. 1998, 63, 199) in methylene chloride to yield formula (3). Fmoc protecting group on formula (3) was deprotected using 20% piperidine in DMF to give the thiourea on a solid support of formula (4). Thiourea (4) is reacted with methyl iodide to produce a compound of formula (5). Reaction of the compound of the formula (5) with amines (6) afforded a compound of formula (7). The compound of formula (7) is reacted with chlorocarbonyl isocyanate in THF to produce a compound of formula (8). Mitsunobu reaction of a compound of formula (8) with alcohol afforded a compound of formula (9) The compound of formula (I) wherein R¹, R², and R³ are as defined above is removed from the solid support with an acidic cleavage mixture such as trifluoroacetic acid in dichloromethane.

Compounds of the present invention are believed to be pharmaceutically active agents having anti-inflammatory properties. Thus, the present invention also provides a pharmaceutical composition which comprises a compound of this invention in combination or association with a pharmaceutically acceptable carrier. In particular, the present invention provides a pharmaceutical composition which comprises an effective amount of a compound of this invention and a pharmaceutically acceptable carrier.

The compositions are preferably adapted for oral administration. However, they may be adapted for other modes of administration, for example, parenteral administration.

In order to obtain consistency of administration, it is preferred that a composition of the invention is in the form of a unit dose. Suitable unit dose forms include tablets, capsules and powders in sachets or vials. Such unit dose forms may contain from 0.1 to 100 mg of a compound of the invention and preferably from 2 to 50 mg. Still further preferred unit dosage forms contain 5 to 25 mg of a compound of the present invention. The compounds of the present invention can be administered orally at a dose range of about 0.01 to 100 mg/kg or preferably at a dose range of 0.1 to 10 mg/kg. Such compositions may be administered from 1 to 6 times a day, more usually from 1 to 4 times a day.

The compositions of the invention may be formulated with conventional excipients, such as a filler, a disintegrating agent, a binder, a lubricant, a flavoring agent and the like. They are formulated in conventional manner, for example, in a manner similar to that use for known analgesics.

### EXAMPLES

The following examples are illustrative and are not meant to be limiting of the present invention.

Abbreviations:

Fmoc: 9-fluorenylmethoxycarbonyl

DMF: N,N-Dimethylformaide

HOBT: N-Hydroxybenzotriazole

DMAP: dimethylaminopyridine

DIC: N,N′-diisopropylcarbodiimide

MeOH: Methanol

DMSO: Dimethylsulfoxide

TMAD: Tetramethylazodicarboxamide

TFA: Trifluoroacetic acid

### Example 1

### Preparation of 4-(3-(4-bromobenzyl)-2,4-dioxo-6-piperidin-1-yl-3,4-dihydro-1,3,5-triazin-1(2H)-1(2H)-yl)benzoic Acid on Wang Resin

Step 1: Fluorenylmethyloxycarbonyl Isothiocyanate

The title compound was prepared from fluorenylmethyloxycarbonyl chloride and potassium thiocyanate according to the procedure of Kearney et al. J. Org. Chem. 1998, 63, 199; ¹H NMR (CDCl₃) δ 7.75 (d, J=7.5 Hz, 2H), 7.56 (d, J=7.5 Hz, 2H), 7.41 (t, J=7.5 Hz, 2H), 7.32 (t, J=7.5 Hz, 2H), 4.44 (d, J=7.4 Hz, 2H), 4.23 (t, J=7.4 Hz, 1H).

IR(cm⁻¹): 1963.32 (N═C═S stretch)

Step 2: Attachment of N-Fmoc-4-Aminobenzoic Acid to Wang Resin

Wang Resin (Wang, S. J. Am. Chem. Soc. 1973, 95, 1328-1333) (Ana Spec 100-200 mesh, 1% crosslinked; loading: 1.1 mmol/g; 5 g, 5.5 mmol) was swollen in anhydrous DMF (20 ml). A solution of N-Fmoc-4-Aminobenzoic acid (7.9 g, 22 mmol), HOBT (3.37 g, 22 mmol), DMAP (268.8 mg, 2.2 mmol) and DIC (3.4 ml, 22 mmol) in anhydrous DMF (30 ml) was added to the resin. The mixture was shaked at room temperature on an orbital shaker overnight. The mixture was filtered and the resin was washed with DMF (3×50 ml), MeOH (3×50 ml), CH₂Cl₂ (3×50 ml), and dried.

Step 3: Deprotection of Fmoc Group

The resin (5.5 mmol), prepared as described in step 2 above, was treated with a solution of 20% piperidine in DMF (2×50 ml, 10 min for the first time and 30 min for the second time) to remove the Fmoc protecting group from the resin. The mixture was filtered and the resin was washed with DMF (3×50 ml), MeOH (3×50 ml), and CH₂Cl₂ (3×50 ml).

Step 4: Reaction with Fmoc-isothiocyanate

To the 4-aminobenzoic acid on Wang resin (5.5 mmol) was added a solution of Fmoc-isothiocyanate (3.09 g, 11 mmol, prepared as described in step 1) in anhydrous CH₂Cl₂ (50 ml). After 20 min, the mixture was filtered, washed with CH₂Cl₂ (5×50 ml).

Step 5: Deprotection of Fmoc Group

The resin (5.5 mmol) obtained from step 4 was reacted again with a solution of 20% piperidine in DMF (2×50 ml, 10 min for the first time and 30 min for the second time) to produce the thiourea. The mixture was filtered and the resin was washed with DMF (3×50 ml), MeOH (3×50 ml), CH₂Cl₂ (3×50 ml), and dried.

To confirm that the reaction occured, 100 mg of resin was treated with 50% TFA/CH₂Cl₂ for 1 hr, filtered, and the filtrate was concentrated. LC/MS analysis, showed correct M⁺+H, 197.

Step 6: Preparation of the Resin-bounded Methyl Thiourea

To the resin-bounded thiourea (5.5 mmol) in anhydrous DMF (50 ml) was added Mel (6.85 ml, 0.11 mol). After half an hour, the mixture was filtered and treated again with equal amount of Mel in DMF overnight. The mixture was then filtered and the resin was washed with DMF (3×50 ml), MeOH (3×50 ml), CH₂Cl₂ (3×50 ml), and dried.

To confirm that the reaction occured, 100 mg of resin was treated with 50% TFA/CH₂Cl₂ for 1 hr, filtered, and the filtrate was concentrated. LC/MS analysis, showed correct M⁺+H, 211.

Step 7: Displacement of the Methylthio Group with Piperidine

A mixture of the resin (200 mg, 0.22 mmol; loading 1.1 mmol/g), prepared as described in step 6 and Piperidine (87 μl, 0.88 mmol) in anhydrous Dimethylsulfoxide (4 ml) was heated at 80° C. on the J-KEM block for 24 hrs. The mixture was then filtered and the resin was washed with DMSO (3×4 ml), MeOH (3×4 ml), CH₂Cl₂ (3×4 ml), and dried.

To confirm that the reaction occured, 100 mg of resin was treated with 50% TFA/CH₂Cl₂ for 1 hr, filtered, and the filtrate was concentrated. LC/MS analysis, showed correct M⁺+H, 248.

Step 8: The Formation of 1,3,5-Triazine-2,4-dione

The resin (200 mg, 0.22 mmol; loading 1.1 mmol/g) obtained from step 7 was swollen in anhydrous THF (4 ml), (70.8 μl, 0.88 mmol) of chlorocarbonylisocyanate was subsequently added. This reaction was allowed to shake at room temperature for 2 hrs. The mixture was filtered and the resin was washed with THF (3×4 ml), CH₂Cl₂ (3×4 ml), and dried.

To confirm that the reaction occured, 100 mg of resin was treated with 50% TFA/CH₂Cl₂ for 1 hr, filtered, and the filtrate was concentrated. LC/MS analysis, showed correct M⁺+H, 317. ¹H NMR (DMSO-d₆) δ 1.21 (m, 4H), 1.38-1.40 (m, 2H), 3.10-3.14 (m, 4H), 7.57 (d, 2H), 8.03 (d, 2H), 11.10 (s, 1H), 13.15 (s, 1H).

Step 9: Mitsnubo Reaction with 4-Bromobenzyl Alcohol

To the resin (200 mg, 0.22 mmol; loading 1.1 mmol/g) in 1:1 THF/CH₂Cl₂ (4 ml) was added tetramethylazodicarboxamide (189.4 mg, 1.1 mmol), followed by 4-bromobenzyl alcohol (205.7 mg, 1.1 mmol). Finally, tributylphosphine (275 μl, 1.1 mmol) was added. The resulting reaction mixture was rotated at room temperature overnight. The mixture was filtered and the resin was washed with THF (3×4 ml), MeOH (3×4 ml), CH₂Cl₂ (3×4 ml) and finally cleaved from resin with 50% TFA/CH₂Cl₂ (4 ml) for 1 hr, filtered, and the filtrate was concentrated to 4-(3-(4-bromobenzyl)-2,4-dioxo-6-piperidin-1-yl-3,4-dihydro-1,3,5-triazin-1(2H)-1(2H)-yl)benzoic acid; LC/MS analysis, showed correct M⁺+H, 485.30. ¹H NMR (DMSO-d₆) δ 1.22-1.23 (m, 4H), 1.39-1.40 (m, 2H), 3.12-3.16 (m, 4H), 4.87 (s, 2H), 7.30 (d, 2H), 7.51 (d, 2H), 7.61 (d, 2H), 8.03 (d, 2H), 13.25 (s, 1H).

### Example 2

### Preparation of 4-(6-[benzyl(ethyl)amino]-3-(4-bromobenzyl)-2,4-dioxo-3,4-dihydro-1,3,5-triazin-1(2H)-yl)benzoic Acid on Wang Resin

The resin product was prepared according to step 7 and step 8 of example 1 from 4-aminobenzoic acid methyl isothiourea on Wang resin and N-ethylbenzylamine followed by the cyclization.

A sample of resin was treated with 50% TFA/CH₂Cl₂ as in step 8 of example 1 to yield 4-(6-[benzyl(ethyl)amino]-2,4-dioxo-3,4-dihydro-1,3,5-triazin-1(2H)-yl)-benzoic acid; ¹H NMR (DMSO-d₆) δ 0.66 (t, 3H), 2.97 (q, 2H), 4.39 (s, 2H), 7.18-7.34 (m, 5H), 7.56 (d, 2H), 7.99 (d, 2H), 11.10 (s, 1H), 13.20 (s, 1H).

The final mitsnubo step (step 9) was performed as detailed in example 1 using 4-bromobenzyl alcohol to give 4-(6-[benzyl(ethyl)amino]-3-(4-bromobenzyl)-2,4-dioxo-3,4-dihydro-1,3,5-triazin-1(2H)-yl)benzoic acid; ¹H NMR (DMSO-d₆) δ 0.64 (t, 3H), 2.99 (q, 2H), 4.43 (s, 2H), 4.88 (s, 2H), 7.19-7.29 (m, 5H), 7.31 (d, 2H), 7.51 (d, 2H), 7.61 (d, 2H), 7.98 (d, 2H), 13.25 (s, 1H).

### Example 3

### Preparation of 4-(3-(4-bromobenzyl)-6-(dipropylamino)-2,4-dioxo-3,4-dihydro-1,3,5-triazin-1(2H)-yl)benzoic Acid on Wang Resin

The resin product was prepared according to step 7 and step 8 of example 1 from 4-aminobenzoic acid methyl isothiourea on Wang resin and dipropylamine followed by the cyclization.

A sample of resin was treated with 50% TFA/CH₂Cl₂ as in step 8 of example 1 to yield 4-(6-(dipropylamino)-2,4-dioxo-3,4-dihydro-1,3,5-triazin-1(2H)-yl)benzoic acid; ¹H NMR (DMSO-d₆) δ 0.64 (t, 6H), 1.18-1.30 (m, 4H), 2.98 (t, 4H), 7.55 (d, 2H), 8.04 (d, 2H), 11.00 (s, 1H), 13.22 (s, 1H).

The final mitsnubo step (step 9) was performed as detailed in example 1 using 4-bromobenzyl alcohol to give 4-(3-(4-bromobenzyl)-6-(dipropylamino)-2,4-dioxo-3,4-dihydro-1,3,5-triazin-1(2H)-yl)benzoic acid; ¹H NMR (DMSO-d₆) δ 0.63 (t, 6H), 1.18-1.30 (m, 4H), 3.00 (t, 4H), 4.86 (s, 2H), 7.29 (d, 2H), 7.50 (d, 2H), 7.59 (d, 2H), 8.04 (d, 2H), 13.28 (s, 1H).

### Example 4

### Preparation of 3-(3-(4-bromobenzyl)-2,4-dioxo-6-piperidin-1-yl-3,4-dihydro-1,3,5-triazin-1(2H)-yl)benzoic Acid on Wang Resin

The resin product was prepared according to step 7 and step 8 of example 1 from 3-aminobenzoic acid methyl isothiourea on Wang resin and Piperidine followed by the cyclization.

A sample of resin was treated with 50% TFA/CH₂Cl₂ as in step 8 of example 1 to yield 3-(2,4-dioxo-6-piperidin-1-yl-3,4-dihydro-1,3,5-triazin-1(2H)-yl)benzoic acid; ¹H NMR (DMSO-d₆) δ 1.18 (m, 4H), 1.37-1.39 (m, 2H), 3.10-3.11 (m, 4H), 7.61 (t, 1H), 7.70 (d, 1H), 7.97 (d, 2H), 11.10 (s, 1H), 13.15 (s, 1H).

The final mitsnubo step (step 9) was performed as detailed in example 1 using 4-bromobenzyl alcohol to give 3-(3-(4-bromobenzyl)-2,4-dioxo-6-piperidin-1-yl-3,4-dihydro-1,3,5-triazin-1(2H)-yl)benzoic acid; ¹H NMR (DMSO-d₆) δ 1.19 (m, 4H), 1.38-1.39 (m, 2H), 3.12-3.15 (m, 4H), 4.86 (s, 2H), 7.30 (d, 2H), 7.50 (d, 2H), 7.62 (t, 1H), 7.73 (d, 1H), 7.97 (d, 1H), 8.05 (d, 1H), 13.30 (s, 1H).

### Example 5

### Preparation of 4-(3-(4-bromobenzyl)-2,4-dioxo-6-piperidin-1-yl-3,4-dihydro-1,3,5-triazin-1(2H)-yl)-2-chlorobenzoic Acid Wang Resin

The resin product was prepared according to step 7 and step 8 of example 1 from 2-chloro-4-aminobenzoic acid methyl isothiourea on Wang resin and Piperidine followed by the cyclization.

A sample of resin was treated with 50% TFA/CH₂Cl₂ as in step 8 of example 1 to yield 2-chloro-4-(2,4-dioxo-6-piperidin-1-yl-3,4-dihydro-1,3,5-triazin-1(2H)-yl)-benzoic acid; ¹H NMR (DMSO-d₆) δ 1.15-1.18 (m, 4H), 1.41-1.43 (m, 2H), 3.11-3.14 (m, 4H), 7.53 (dd, 1H), 7.75 (d, 1H), 7.89 (d, 1H), 11.10 (s, 1H), 13.10 (s, 1H).

The final mitsnubo step (step 9) was performed as detailed in example 1 using 4-bromobenzyl alcohol to give 4-(3-(4-bromobenzyl)-2,4-dioxo-6-piperidin-1-yl-3,4-dihydro-1,3,5-triazin-1(2H)-yl)-2-chlorobenzoic acid; ¹H NMR (DMSO-d₆) δ 1.26 (m, 4H), 1.42-1.43 (m, 2H), 3.14-3.17 (m, 4H), 4.86 (s, 2H), 7.30 (d, 2H), 7.51 (d, 2H), 7.57 (dd, 1H), 7.80 (d, 1H), 7.90 (d, 1H), 13.30 (s, 1H).

### Example 6

### Preparation of 5-(3-(4-bromobenzyl)-2,4-dioxo-6-piperidin-1-yl-3,4-dihydro-1,3,5-triazin-1(2H)-yl)-2-chlorobenzoic Acid on Wang Resin

The resin product was prepared according to step 7 and step 8 of example 1 from 2-chloro-5-aminobenzoic acid methyl isothiourea on Wang resin and Piperidine followed by the cyclization.

A sample of resin was treated with 50% TFA/CH₂Cl₂ as in step 8 of example 1 to yield 2-chloro-5-(2,4-dioxo-6-piperidin-1-yl-3,4-dihydro-1,3,5-triazin-1(2H)-yl)-benzoic acid; ¹H NMR (DMSO-d₆) δ 1.23 (m, 4H), 1.40-1.41 (m, 2H), 3.10-3.11 (m, 4H), 7.61-7.69 (m, 2H), 7.90 (d, 1H), 11.10 (s, 1H), 13.10 (s, 1H).

The final mitsnubo step (step 9) was performed as detailed in example 1 using 4-bromobenzyl alcohol to give 5-(3-(4-bromobenzyl)-2,4-dioxo-6-piperidin-1-yl-3,4-dihydro-1,3,5-triazin-1(2H)-yl)-2-chlorobenzoic acid; ¹H NMR (DMSO-d₆) δ 1.12-1.24 (m, 4H), 1.41-1.42 (m, 2H), 3.05-3.14 (m, 4H), 4.86 (s, 2H), 7.30 (d, 2H), 7.50 (d, 2H), 7.68 (m, 2H), 7.97 (d, 1H), 13.20 (s, 1H).

### Example 7

### Preparation of 4-(2,4-dioxo-6-piperidin-1-yl-3-[4-(trifluoromethyl)benzyl]-3,4-dihydro-1,3,5-triazin-1(2H)-yl)benzoic Acid on Wang Resin

The resin product was prepared according to step 7 through step 9 of example 1 from 4-aminobenzoic acid methyl isothiourea on Wang resin and piperidine followed by the cyclization and finally mitsnubo reaction using p-trifluoromethylbenzyl alcohol.

A sample of resin was treated with 50% TFA/CH₂Cl₂ as in step 9 of example 1 to yield 4-(2,4-dioxo-6-piperidin-1-yl-3-[4-(trifluoromethyl)benzyl]-3,4-dihydro-1,3,5-triazin-1(2H)-yl)benzoic acid; ¹H NMR (DMSO-d₆) δ 1.23-1.24 (m, 4H), 1.40-1.41 (m, 2H), 3.14-1.17 (m, 4H), 4.99 (s, 2H), 7.55 (d, 2H), 7.62 (d, 2H), 7.68 (d, 2H), 8.03 (d, 2H), 13.21 (s, 1H).

### Example 8

### Preparation of 4-(3-[4-(benzyloxy)benzyl]-2,4-dioxo-6-piperidin-1-yl-3,4-dihydro-1,3,5-triazin-1(2H)-yl)benzoic Acid on Wang Resin

The resin product was prepared according to step 7 through step 9 of example 1 from 4-aminobenzoic acid methyl isothiourea on Wang resin and piperidine followed by the cyclization and finally mitsnubo reaction using p-benzyloxybenzyl

A sample of resin was treated with 50% TFA/CH₂Cl₂ as in step 9 of example 1 to yield 4-(3-[4-(benzyloxy)benzyl]-2,4-dioxo-6-piperidin-1-yl-3,4-dihydro-1,3,5-triazin-1(2H)-yl)benzoic acid; ¹H NMR (DMSO-d₆) δ 1.21 (m, 4H), 1.38-1.39 (m, 2H), 3.11-3.14 (m, 4H), 4.83 (s, 2H), 5.07 (s, 2H), 6.93 (d, 2H), 7.27 (d, 2H), 7.31-7.44 (m, 5H), 7.60 (d, 2H), 8.03 (d, 2H), 13.20 (s, 1H).

### Example 9

### Preparation of 4-(3-(3,4-dimethylbenzyl)-2,4-dioxo-6-piperidin-1-yl-3,4-dihydro-1,3,5-triazin-1(2H)-yl)benzoic Acid on Wang Resin

The resin product was prepared according to step 7 through step 9 of example 1 from 4-aminobenzoic acid methyl isothiourea on Wang resin and piperidine followed by the cyclization and finally mitsnubo reaction using 3,4-dimethyl-benzylalcohol.

A sample of resin was treated with 50% TFA/CH₂Cl₂ as in step 9 of example 1 to yield 4-(3-(3,4-dimethylbenzyl)-2,4-dioxo-6-piperidin-1-yl-3,4-dihydro-1,3,5-triazin-1(2H)-yl)benzoic acid; ¹H NMR (DMSO-d₆) δ 1.21-1.22 (m, 4H), 1.29-1.32 (m, 2H), 2.17 (s, 6H), 3.12-3.16 (m, 4H), 4.82 (s, 2H), 7.04 (d, 2H), 7.09 (s, 1H), 7.60 (d, 2H), 8.02 (d, 2H), 13.22 (s, 1H)

### Example 10

### Preparation of 4-(3-([1,1′-biphenyl]-2-ylmethyl)-2,4-dioxo-6-piperidin-1-yl-3,4-dihydro-1,3,5-triazin-1(2H)-yl)benzoic Acid on Wang Resin

The resin product was prepared according to step 7 through step 9 of example 1 from 4-aminobenzoic acid methyl isothiourea on Wang resin and piperidine followed by the cyclization and finally mitsnubo reaction using 2-biphenylmethanol.

A sample of resin was treated with 50% TFA/CH₂Cl₂ as in step 9 of example 1 to yield 4-(3-([1,1′-biphenyl]-2-ylmethyl)-2,4-dioxo-6-piperidin-1-yl-3,4-dihydro-1,3,5-triazin-1(2H)-yl)benzoic acid; ¹H NMR (DMSO-d₆) δ 1.21 (m, 4H), 1.40-1.41 (m, 2H), 3.14-3.15 (m, 4H), 4.86 (s, 2H), 7.20 (m, 2H), 7.30-7.46 (m, 7H), 7.57 (d, 2H), 8.02 (d, 2H), 13.25 (s, 1H).

### Example 11

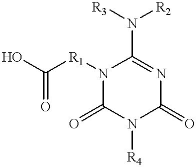

### Parallel Synthesis of Sixty Compounds

Sixty compounds were synthesized in parallel. Twelve scintillation vials were first arranged in a 4×3 matrix. One of each of the resins prepared according to the procedure outlined in Example 1 was placed in 3 scintillation vials in one row (1 g per vial, 1.1 mmol; loading 1.1 mmol/g): 2-Cl-4-aminobenzoic acid on Wang resin in row 1; 2-Cl-5-aminobenzoic acid on Wang resin in row 2; 4-aminobenzoic acid on Wang resin in row 3; 3-aminobenzoic acid on Wang resin in row 4. To each vial was added secondary amines in anhydrous Dimethylsulfoxide: Piperidine in column 1; N-ethylbenzylamine in column 2; Dipropylamine in column 3. The reaction vials were then placed in a J-KEM block and rotated at 80° C. for 24 hrs. The reaction mixtures were transferred into twelve filtration vessels in a multiple peptide synthesizer. The mixtures were filtered and the resin in each vessel was washed with dimethylsulfoxide (3×10 ml), methanol (3×10 ml), dichloromethane (3×10 ml), and dried. To the resin in each reaction vessel obtained above in anhydrous THF (4 ml), chlorocarbonylisocyanate was added. The mixtures were shaked at room temperature for 2 hrs. The resulting products on the resin were filtered and washed with tetrahydrofuran (3×10 ml) and dichloromethane (3×10 ml), and dried. 1 g resin in each of the twelve reaction vessels as prepared above was further divided into 5 smaller vessels (200 mg per vessel, 0.22 mmol; loading 1.1 mmol/g). To each vessel with resin swollen in 1:1 THF/DCM was added tetramethylazodicarboxamide, followed with five different benzyl alcohols: 4-Bromobenzyl alcohol, P-trifluoromethylbenzyl alcohol, P-benzyloxybenzyl alcohol, 3,4-Dimethylbenzyl alcohol and 2-Biphenylmethanol, respectively. Finally, tributylphosphine was added. The sixty reaction vessels were rotated at room temperature overnight. The resulting mixtures on the resin were filtered and washed with tetrahydrofuran (3×4 ml), methanol (3×4 ml), dichloromethane (3×4 ml), and dried.

The 60 products were cleaved from the solid support for characterization according to the following procedure. To each vessel was added 1:1 trifluoroacetic acid/dichloromethane (4 ml). The synthesizer was left standing for 1 h, and the solution were filtered into 1 dram vials. The resin in each vessel was washed with dichloromethane (2 ml). The solutions were concentrated under a nitrogen stream and dried in Savant under vacuum. The characterization datas were listed in the following table.

Solid Phase Synthesis of 1,3,5-Triazine-2,4-Dione Derivatives

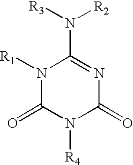

| | | | | | LC (min) | Isolated |
|---|---|---|---|---|---|---|
| Ex. | R₁ | R₂ | R₃ | R₄ | MS (M + H) | Yield (mg) |
| 12 | 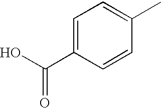 | Piperidine | | 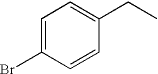 | 2.793 485.0 | 13.0 |
| 13 | 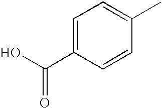 | ethyl | benzyl | 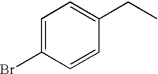 | 3.147 535.1 | 15.1 |
| 14 | 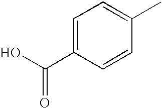 | propyl | propyl | 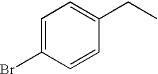 | 3.101 501.1 | 16.8 |
| 15 | 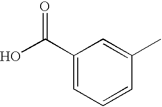 | Piperidine | | 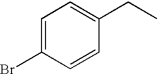 | 2.959 485.30 | 17.8 |
| 16 | 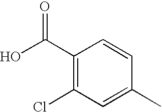 | Piperidine | | 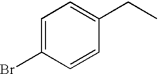 | 2.926 519.0 | 11.2 |
| 17 | 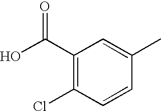 | Piperidine | | 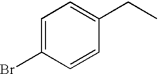 | 2.957 519.0 | 30.3 |
| 18 | 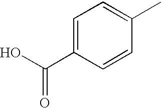 | Piperidine | | 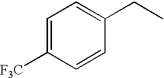 | 2.883 475.15 | 11.6 |
| 19 | 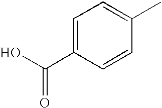 | Piperidine | | 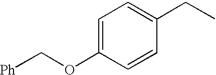 | 3.102 513.2 | 10.6 |
| 20 | 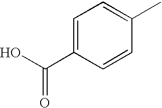 | Piperidine | | 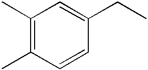 | 2.793 435.2 | 9.4 |
| 21 | 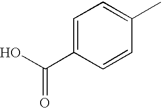 | Piperidine | | 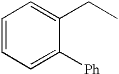 | 2.971 483.2 | 10.5 |
| 22 | 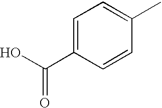 | ethyl | benzyl | 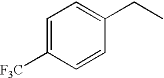 | 3.218 525.15 | 12.3 |
| 23 | 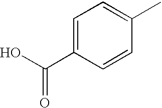 | ethyl | benzyl | 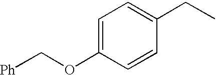 | 3.401 563.2 | 11.2 |
| 24 | 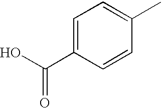 | ethyl | benzyl | 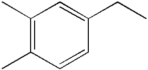 | 3.143 485.2 | 12.2 |
| 25 | 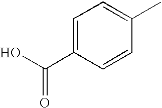 | ethyl | benzyl | 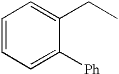 | 3.312 533.2 | 11.0 |
| 26 | 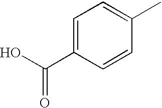 | propyl | propyl | 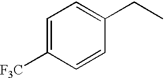 | 3.170 491.2 | 13.8 |
| 27 | 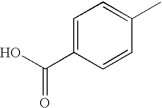 | propyl | propyl | 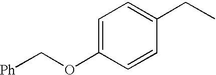 | 3.364 529.2 | 14.5 |
| 28 | 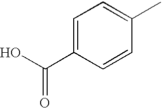 | propyl | propyl | 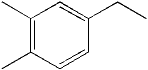 | 3.097 451.2 | 14.0 |
| 29 | 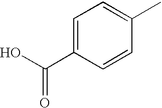 | propyl | propyl | 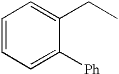 | 3.261 499.2 | 13.1 |
| 30 | 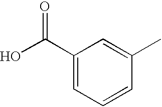 | Piperidine | | 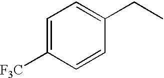 | 2.981 475.38 | 14.6 |
| 31 | 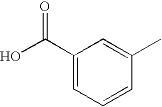 | Piperidine | | 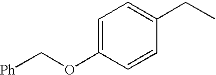 | 3.157 513.47 | 19.8 |
| 32 | 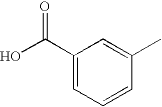 | Piperidine | | 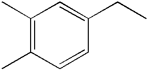 | 2.918 435.42 | 17.9 |
| 33 | 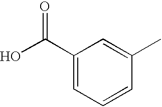 | Piperidine | | 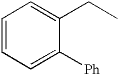 | 2.885 483.43 | 15.9 |
| 34 | 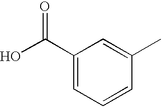 | ethyl | benzyl | 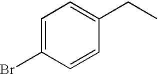 | 3.153 535.1 | 24.5 |
| 35 | 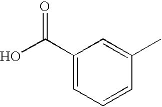 | ethyl | benzyl | 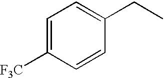 | 3.218 525.15 | 29.5 |
| 36 | 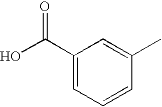 | ethyl | benzyl | 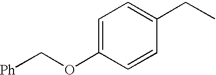 | 3.047 563.2 | 10.9 |
| 37 | 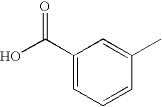 | ethyl | benzyl | 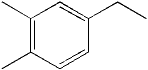 | 3.149 485.2 | 21.6 |
| 38 | 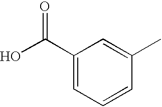 | ethyl | benzyl | 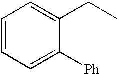 | 3.303 533.2 | 25.2 |
| 39 | 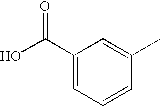 | propyl | propyl | 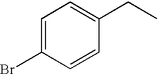 | 3.112 501.1 | 28.1 |
| 40 | 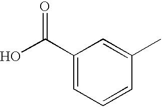 | propyl | propyl | 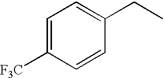 | 3.190 491.2 | 29.5 |
| 41 | 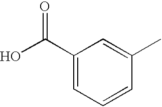 | propyl | propyl | 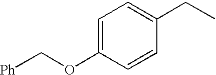 | 3.009 529.2 | 9.6 |
| 42 | 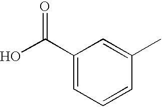 | proyl | propyl | 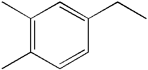 | 3.114 451.2 | 22.8 |
| 43 | 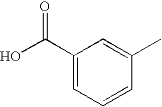 | propyl | propyl | 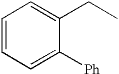 | 3.266 499.2 | 21.9 |
| 44 | 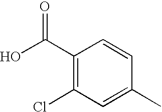 | Piperidine | | 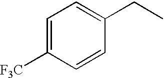 | 2.998 509.1 | 9.9 |
| 45 | 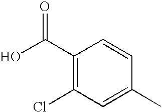 | Piperidine | | 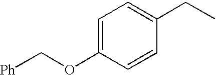 | 3.211 547.2 | 8.2 |
| 46 | 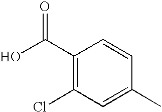 | Piperidine | | 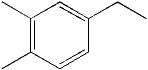 | 2.926 469.15 | 8.8 |
| 47 | 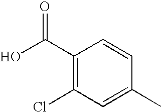 | Piperidine | | 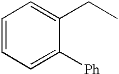 | 3.094 517.15 | 8.3 |
| 48 | 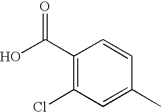 | ethyl | benzyl | 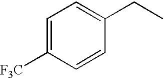 | 3.248 569.0 | 12.7 |
| 49 | 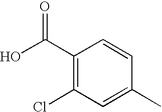 | ethyl | benzyl | 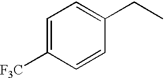 | 3.303 559.1 | 12.2 |
| 50 | 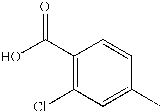 | ethyl | benzyl | 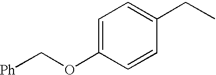 | 3.482 597.2 | 9.3 |
| 51 | 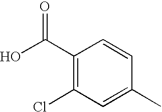 | ethyl | benzyl | 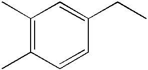 | 3.246 519.15 | 11.5 |
| 52 | 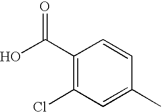 | ethyl | benzyl | 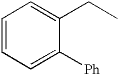 | 3.397 567.2 | 6.4 |
| 53 | 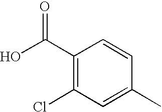 | propyl | propyl | 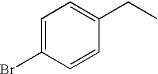 | 3.094 517.15 | 8.7 |
| 54 | 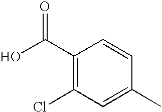 | propyl | propyl | 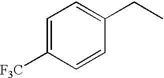 | 3.280 525.1 | 8.8 |
| 55 | 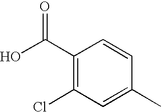 | propyl | propyl | 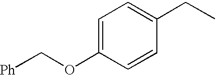 | 3.075 563.2 | 3.2 |
| 56 | 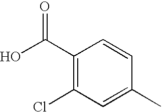 | propyl | propyl | 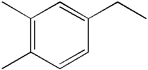 | 3.222 485.2 | 9.4 |
| 57 | 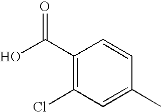 | propyl | propyl | 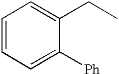 | 3.371 533.2 | 4.7 |
| 58 | 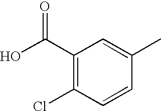 | Piperidine | | 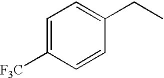 | 3.040 509.1 | 26.2 |
| 59 | 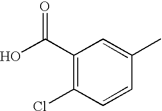 | Piperidine | | 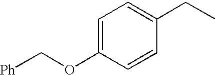 | 3.252 547.2 | 19.6 |
| 60 | 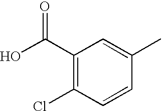 | Piperidine | | 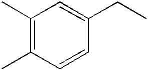 | 2.953 469.15 | 26.5 |
| 61 | 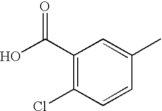 | Piperidine | | 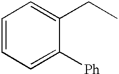 | 3.117 517.1 | 19.8 |
| 62 | 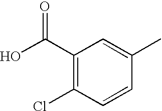 | ethyl | benzyl | 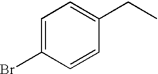 | 3.272 569.0 | 32.4 |
| 63 | 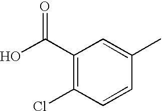 | ethyl | benzyl | 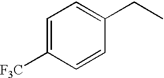 | 3.338 559.1 | 33.0 |
| 64 | 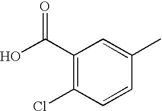 | ethyl | benzyl | 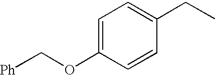 | 3.518 597.2 | 29.3 |
| 65 | 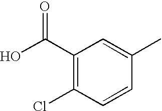 | ethyl | benzyl | 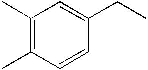 | 3.277 519.2 | 30.0 |
| 66 | 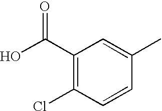 | ethyl | benzyl | 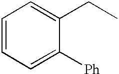 | 3.421 567.2 | 27.1 |
| 67 | 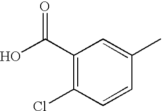 | propyl | propyl | 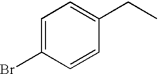 | 3.248 535.1 | 30.5 |
| 68 | 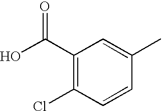 | propyl | propyl | 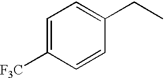 | 3.310 525.1 | 30.8 |
| 69 | 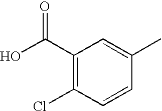 | propyl | propyl | 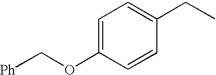 | 3.501 563.2 | 28.4 |
| 70 | 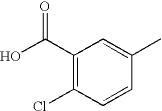 | propyl | propyl | 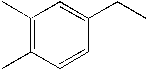 | 3.249 485.2 | 25.1 |
| 71 | 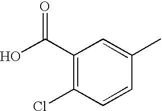 | propyl | propyl | 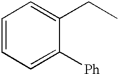 | 3.391 533.2 | 23.9 |

### Example 12

4-(3-(4-bromobenzyl)-2,4-dioxo-6-piperidin-1-yl-3,4-dihydro-1,3,5-triazin-1(2H)-yl)benzoic acid

### Example 13

4-(6-[benzyl(ethyl)amino]-3-(4-bromobenzyl)-2,4-dioxo-3,4-dihydro-1,3,5-triazin-1(2H)-yl)benzoic acid

### Example 14

4-(3-(4-bromobenzyl)-6-(dipropylamino)-2,4-dioxo-3,4-dihydro-1,3,5-triazin-1(2H)-yl)benzoic acid

### Example 15

3-(3-(4-bromobenzyl)-2,4-dioxo-6-piperidin-1-yl-3,4-dihydro-1,3,5-triazin-1(2H)-yl)benzoic acid

### Example 16

4-(3-(4-bromobenzyl)-2,4-dioxo-6-piperidin-1-yl-3,4-dihydro-1,3,5-triazin-1(2H)-yl)-2-chlorobenzoic acid

### Example 17

5-(3-(4-bromobenzyl)-2,4-dioxo-6-piperidin-1-yl-3,4-dihydro-1,3,5-triazin-1(2H)-yl)-2-chlorobenzoic acid

### Example 18

4-(2,4-dioxo-6-piperidin-1-yl-3-[4-(trifluoromethyl)benzyl]-3,4-dihydro-1,3,5-triazin-1(2H)-yl)benzoic acid

### Example 19

4-(3-[4-(benzyloxy)benzyl]-2,4-dioxo-6-piperidin-1-yl-3,4-dihydro-1,3,5-triazin-1(2H)-yl)benzoic acid

### Example 20

4-(3-(3,4-dimethylbenzyl)-2,4-dioxo-6-piperidin-1-yl-3,4-dihydro-1,3,5-triazin-1(2H)-yl)benzoic acid

### Example 21

4-(3-([1,1′-biphenyl]-2-ylmethyl)-2,4-dioxo-6-piperidin-1-yl-3,4-dihydro-1,3,5-triazin-1(2H)-yl)benzoic acid

### Example 22

4-(6-[benzyl(ethyl)amino]-2,4-dioxo-3-[4-(trifluoromethyl)benzyl]-3,4-dihydro-1,3,5-triazin-1(2H)-yl)benzoic acid

### Example 23

4-(6-[benzyl(ethyl)amino]-3-[4-(benzyloxy)benzyl]-2,4-dioxo-3,4-dihydro-1,3,5-triazin-1(2H)-yl)benzoic acid

### Example 24

4-(6-[benzyl(ethyl)amino]-3-(3,4-dimethylbenzyl)-2,4-dioxo-3,4-dihydro-1,3,5-triazin-1(2H)-yl)benzoic acid

### Example 25

4-(6-[benzyl(ethyl)amino]-3-([1,1′-biphenyl]-2-ylmethyl)-2,4-dioxo-3,4-dihydro-1,3,5-triazin-1(2H)-yl)benzoic acid

### Example 26

4-(6-(dipropylamino)-2,4-dioxo-3-[4-(trifluoromethyl)benzyl]-3,4-dihydro-1,3,5-triazin-1(2H)-yl)benzoic acid

### Example 27

4-(3-[4-(benzyloxy)benzyl]-6-(dipropylamino)-2,4-dioxo-3,4-dihydro-1,3,5-triazin-1(2H)-yl)benzoic acid

### Example 28

4-(3-(3,4-dimethylbenzyl)-6-(dipropylamino)-2,4-dioxo-3,4-dihydro-1,3,5-triazin-1(2H)-yl)benzoic acid

### Example 29

4-(3-([1,1′-biphenyl]-2-ylmethyl)-6-(dipropylamino)-2,4-dioxo-3,4-dihydro-1,3,5-triazin-1(2H)-yl)benzoic acid

### Example 30

3-(2,4-dioxo-6-piperidin-1-yl-3-[4-(trifluoromethyl)benzyl]-3,4-dihydro-1,3,5-triazin-1(2H)-yl)benzoic acid

### Example 31

3-(3-[4-(benzyloxy)benzyl]-2,4-dioxo-6-piperidin-1-yl-3,4-dihydro-1,3,5-triazin-1(2H)-yl)benzoic acid

### Example 32

3-(3-(3,4-dimethylbenzyl)-2,4-dioxo-6-piperidin-1-yl-3,4-dihydro-1,3,5-triazin-1(2H)-yl)benzoic acid

### Example 33

3-(3-([1,1′-biphenyl]-2-ylmethyl)-2,4-dioxo-6-piperidin-1-yl-3,4-dihydro-1,3,5-triazin-1(2H)-yl)benzoic acid

### Example 34

3-(6-[benzyl(ethyl)amino]-3-(4-bromobenzyl)-2,4-dioxo-3,4-dihydro-1,3,5-triazin-1(2H)-yl)benzoic acid

### Example 35

3-(6-[benzyl(ethyl)amino]-2,4-dioxo-3-[4-(trifluoromethyl)benzyl]-3,4-dihydro-1,3,5-triazin-1(2H)-yl)benzoic acid

### Example 36

3-(6-[benzyl(ethyl)amino]-3-[4-(benzyloxy)benzyl]-2,4-dioxo-3,4-dihydro-1,3,5-triazin-1(2H)-yl)benzoic acid

### Example 37

3-(6-[benzyl(ethyl)amino]-3-(3,4-dimethylbenzyl)-2,4-dioxo-3,4-dihydro-1,3,5-triazin-1(2H)-yl)benzoic acid

### Example 38

3-(6-[benzyl(ethyl)amino]-3-([1,1′-biphenyl]-2-ylmethyl)-2,4-dioxo-3,4-dihydro-1,3,5-triazin-1(2H)-yl)benzoic acid

### Example 39

3-(3-(4-bromobenzyl)-6-(dipropylamino)-2,4-dioxo-3,4-dihydro-1,3,5-triazin-1(2H)-yl)benzoic acid

### Example 40

3-(6-(dipropylamino)-2,4-dioxo-3-[4-(trifluoromethyl)benzyl]-3,4-dihydro-1,3,5-triazin-1(2H)-yl)benzoic acid

### Example 41

3-(3-[4-(benzyloxy)benzyl]-6-(dipropylamino)-2,4-dioxo-3,4-dihydro-1,3,5-triazin-1(2H)-yl)benzoic acid

### Example 42

3-(3-(3,4-dimethylbenzyl)-6-(dipropylamino)-2,4-dioxo-3,4-dihydro-1,3,5-triazin-1(2H)-yl)benzoic acid

### Example 43

3-(3-([1,1′-biphenyl]-2-ylmethyl)-6-(dipropylamino)-2,4-dioxo-3,4-dihydro-1,3,5-triazin-1(2H)-yl)benzoic acid

### Example 44

2-chloro-4-(2,4-dioxo-6-piperidin-1-yl-3-[4-(trifluoromethyl)benzyl]-3,4-dihydro-1,3,5-triazin-1(2H)-yl)benzoic acid

### Example 45

4-(3-[4-(benzyloxy)benzyl]-2,4-dioxo-6-piperidin-1-yl-3,4-dihydro-1,3,5-triazin-1(2H)-yl)-2-chlorobenzoic acid

### Example 46

2-chloro-4-(3-(3,4-dimethylbenzyl)-2,4-dioxo-6-piperidin-1-yl-3,4-dihydro-1,3,5-triazin-1(2H)-yl)benzoic acid

### Example 47

4-(3-([1,1′-biphenyl]-2-ylmethyl)-2,4-dioxo-6-piperidin-1-yl-3,4-dihydro-1,3,5-triazin-1(2H)-yl)-2-chlorobenzoic acid

### Example 48

4-(6-[benzyl(ethyl)amino]-3-(4-bromobenzyl)-2,4-dioxo-3,4-dihydro-1,3,5-triazin-1(2H)-yl)-2-chlorobenzoic acid

### Example 49

4-(6-[benzyl(ethyl)amino]-2,4-dioxo-3-[4-(trifluoromethyl)benzyl]-3,4-dihydro-1,3,5-triazin-1(2H)-yl)-2-chlorobenzoic acid

### Example 50

4-(6-[benzyl(ethyl)amino]-3-[4-(benzyloxy)benzyl]-2,4-dioxo-3,4-dihydro-1,3,5-triazin-1(2H)-yl)-2-chlorobenzoic acid

### Example 51

4-(6-[benzyl(ethyl)amino]-3-(3,4-dimethylbenzyl)-2,4-dioxo-3,4-dihydro-1,3,5-triazin-1(2H)-yl)-2-chlorobenzoic acid

### Example 52

4-(6-[benzyl(ethyl)amino]-3-([1,1′-biphenyl]-2-ylmethyl)-2,4-dioxo-3,4-dihydro-1,3,5-triazin-1(2H)-yl)-2-chlorobenzoic acid

### Example 53

4-(3-(4-bromobenzyl)-6-(dipropylamino)-2,4-dioxo-3,4-dihydro-1,3,5-triazin-1(2H)-yl)-2-chlorobenzoic acid

### Example 54

2-chloro-4-(6-(dipropylamino)-2,4-dioxo-3-[4-(trifluoromethyl)benzyl]-3,4-dihydro-1,3,5-triazin -1(2H)-yl)benzoic acid

### Example 55

4-(3-[4-(benzyloxy)benzyl]-6-(dipropylamino)-2,4-dioxo-3,4-dihydro-1,3,5-triazin-1(2H)-yl)-2-chlorobenzoic acid

### Example 56

2-chloro-4-(3-(3,4-dimethylbenzyl)-6-(dipropylamino)-2,4-dioxo-3,4-dihydro-1,3,5-triazin-1(2H)-yl)benzoic acid

### Example 57

4-(3-([1,1′-biphenyl]-2-ylmethyl)-6-(dipropylamino)-2,4-dioxo-3,4-dihydro-1,3,5-triazin-1(2H)-yl)-2-chlorobenzoic acid

### Example 58

2-chloro-5-(2,4-dioxo-6-piperidin-1-yl-3-[4-(trifluoromethyl)benzyl]-3,4-dihydro-1,3,5-triazin-1(2H)-yl)benzoic acid

### Example 59

5-(3-[4-(benzyloxy)benzyl]-2,4-dioxo-6-piperidin-1-yl-3,4-dihydro-1,3,5-triazin-1(2H)-yl)-2-chlorobenzoic acid

### Example 60

2-chloro-5-(3-(3,4-dimethylbenzyl)-2,4-dioxo-6-piperidin-1-yl-3,4-dihydro-1,3,5-triazin-1(2H)-yl)benzoic acid

### Example 61

5-(3-([1,1′-biphenyl]-2-ylmethyl)-2,4-dioxo-6-piperidin-1-yl-3,4-dihydro-1,3,5-triazin-1(2H)-yl)-2-chlorobenzoic acid

### Example 62

5-(6-[benzyl(ethyl)amino]-3-(4-bromobenzyl)-2,4-dioxo-3,4-dihydro-1,3,5-triazin-1(2H)-yl)-2-chlorobenzoic acid

### Example 63

5-(6-[benzyl(ethyl)amino]-2,4-dioxo-3-[4-(trifluoromethyl)benzyl]-3,4-dihydro-1,3,5-triazin-1(2H)-yl)-2-chlorobenzoic acid

### Example 64

5-(6-[benzyl(ethyl)amino]-3-[4-(benzyloxy)benzyl]-2,4-dioxo-3,4-dihydro-1,3,5-triazin-1(2H)-yl)-2-chlorobenzoic acid

### Example 65

5-(6-[benzyl(ethyl)amino]-3-(3,4-dimethylbenzyl)-2,4-dioxo-3,4-dihydro-1,3,5-triazin-1(2H)-yl)-2-chlorobenzoic acid

### Example 66

5-(6-[benzyl(ethyl)amino]-3-([1,1′-biphenyl]-2-ylmethyl)-2,4-dioxo-3,4-dihydro-1,3,5-triazin-1(2H)-yl)-2-chlorobenzoic acid

### Example 67

5-(3-(4-bromobenzyl)-6-(dipropylamino)-2,4-dioxo-3,4-dihydro-1,3,5-triazin-1(2H)-yl)-2-chlorobenzoic acid

### Example 68

2-chloro-5-(6-(dipropylamino)-2,4-dioxo-3-[4-(trifluoromethyl)benzyl]-3,4-dihydro-1,3,5-triazin-1(2H)-yl)benzoic acid

### Example 69

5-(3-[4-(benzyloxy)benzyl]-6-(dipropylamino)-2,4-dioxo-3,4-dihydro-1,3,5-triazin-1(2H)-yl)-2-chlorobenzoic acid

### Example 70

2-chloro-5-(3-(3,4-dimethylbenzyl)-6-(dipropylamino)-2,4-dioxo-3,4-dihydro-1,3,5-triazin-1(2H)-yl)benzoic acid

### Example 71

5-(3-([1,1′-biphenyl]-2-ylmethyl)-6-(dipropylamino)-2,4-dioxo-3,4-dihydro-1,3,5-triazin-1(2H)-yl)-2-chlorobenzoic acid

We claim:

1. A compound having the formula (I) 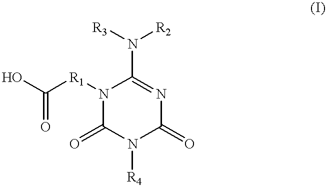
wherein:
R¹ is
an amino acid side chain or phenyl optionally substituted with one or two substituents selected from halogen, straight chain alkyl of 1 to 6 carbon atoms, branched chain alkyl of 2 to 6 carbon atoms, or alkoxy of 1 to 6 carbon atoms;
R² and R³ are independently
hydrogen, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms, phenylalkyl of 7 to 12 carbon atoms, phenyl or acyl; or R² and R³ taken together with the atom to which they are attached, form a 5 or 6 membered heterocyclic ring having one to three heteroatoms selected from O, N and S;
R⁴ is
hydrogen, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms, phenylalkyl of 7 to 12 carbon atoms wherein the phenyl group may be optionally substituted with 1 or 2 substituents selected from
halogen, trifluoromethyl, straight chain or branched alkoxy of 2 to 7 carbon atoms, phenylalkoxy of 7 to 12 carbon atoms, nitro, methylenedioxy, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms or two substituents on adjacent carbons taken together with the phenyl ring form naphthyl optionally substituted with 1 or 2 substituents selected from
halogen, straight chain or branched alkoxy of 2 to 7 carbon atoms, nitro, methylenedioxy, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms; or a pharmaceutical salt thereof.

2. A compound of claim 1 wherein R¹ is is phenyl optionally substituted with halogen.

3. A compound of claim 1 wherein R² and R³, taken together form piperidine.

4. A compound of claim 1 wherein R² is alkyl and R³ is alkyl or phenylalkyl.

5. A compound of claim 1 wherein R⁴ is phenylalkyl.

6. A compound of claim 1 wherein R¹ is phenyl, R² and R³, taken together form a heterocyclic ring, and R⁴ is phenylalkyl.

7. A compound of claim 1 wherein R¹ is phenyl and R⁴ is phenylalkyl.

8. A combinatorial library comprising a plurality of different trisubstituted 1,3,5-triazine-3,3-dione compounds and derivatives thereof according to the formula (I): 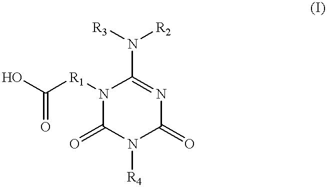
wherein:
R¹ is
amino acid side chain or phenyl optionally substituted with one or two substituents selected from halogen, straight chain alkyl of 1 to 6 carbon atoms, branched chain alkyl of 2 to 6 carbon atoms, or alkoxy of 1 to 6 carbon atoms;
R² and R³ are independently
hydrogen, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms, phenylalkyl of 7 to 12 carbon atoms, phenyl or acyl; or R² and R³ taken together with the atom to which they are attached, form a 5 or 6 membered heterocyclic ring having one to three heteroatoms selected from O, N and S;
R⁴ is
hydrogen, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms, phenylalkyl of 7 to 12 carbon atoms wherein the phenyl group may be optionally substituted with 1 or 2 substituents selected from
halogen, trifluoromethyl, straight chain or branched alkoxy of 2 to 7 carbon atoms, phenylalkoxy of 7 to 12 carbon atoms, nitro, methylenedioxy, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms or two substituents on adjacent carbons taken together with the phenyl ring form naphthyl optionally substituted with 1 or 2 substituents selected from
halogen, straight chain or branched alkoxy of 2 to 7 carbon atoms, nitro, methylenedioxy, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms.

9. The library of claim 8 wherein the compounds are on solid support.

10. A compound of claim 1 which is
4-(3-(4-bromobenzyl)-2,4-dioxo-6-piperidin-1-yl-3,4-dihydro-1,3,5-triazin-1(2H)-yl)benzoic acid;
4-(6-[benzyl(ethyl)amino]-3-(4-bromobenzyl)-2,4-dioxo-3,4-dihydro-1,3,5-triazin-1(2H)-yl)benzoic acid;
4-(3-(4-bromobenzyl)-6-(dipropylamino)-2,4-dioxo-3,4-dihydro-1,3,5-triazin-1(2H)-yl)benzoic acid;
3-(3-(4-bromobenzyl)-2,4-dioxo-6-piperidin-1-yl-3,4-dihydro-1,3,5-triazin-1(2H)-yl)benzoic acid;
4-(3-(4-bromobenzyl)-2,4-dioxo-6-piperidin-1-yl-3,4-dihydro-1,3,5-triazin-1(2H)-yl)-2-chlorobenzoic acid;
5-(3-(4-bromobenzyl)-2,4-dioxo-6-piperidin-1-yl-3,4-dihydro-1,3,5-triazin-1(2H)-yl)-2-chlorobenzoic acid;
4-(2,4-dioxo-6-piperidin-1-yl-3-[4-(trifluoromethyl)benzyl]-3,4-dihydro-1,3,5-triazin-1(2H)-yl)benzoic acid;
4-(3-[4-(benzyloxy)benzyl]-2,4-dioxo-6-piperidin-1-yl-3,4-dihydro-1,3,5-triazin-1(2H)-yl)benzoic acid;
4-(3-(3,4-dimethylbenzyl)-2,4-dioxo-6-piperidin-1-yl-3,4-dihydro-1,3,5-triazin-1(2H)-yl)benzoic acid;
4-(3-([1,1′-biphenyl]-2-ylmethyl)-2,4-dioxo-6-piperidin-1-yl-3,4-dihydro-1,3,5-triazin-1(2H)-yl)benzoic acid;
4-(6-[benzyl(ethyl)amino]-2,4-dioxo-3-[4-(trifluoromethyl)benzyl]-3,4-dihydro-1,3,5-triazin-1(2H)-yl)benzoic acid;
4-(6-[benzyl(ethyl)amino]-3-[4-(benzyloxy)benzyl]-2,4-dioxo-3,4-dihydro-1,3,5-triazin-1(2H)-yl)benzoic acid;
4-(6-[benzyl(ethyl)amino]-3-(3,4-dimethylbenzyl)-2,4-dioxo-3,4-dihydro-1,3,5-triazin-1(2H)-yl)benzoic acid;
4-(6-[benzyl(ethyl)amino]-3-([1,1′-biphenyl]-2-ylmethyl)-2,4-dioxo-3,4-dihydro-1,3,5-triazin-1(2H)-yl)benzoic acid;
4-(6-(dipropylamino)-2,4-dioxo-3-[4-(trifluoromethyl)benzyl]-3,4-dihydro-1,3,5-triazin-1(2H)-yl)benzoic acid;
4-(3-[4-(benzyloxy)benzyl]-6-(dipropylamino)-2,4-dioxo-3,4-dihydro-1,3,5-triazin-1(2H)-yl)benzoic acid;
4-(3-(3,4-dimethylbenzyl)-6-(dipropylamino)-2,4-dioxo-3,4-dihydro-1,3,5-triazin-1(2H)-yl)benzoic acid;
4-(3-([1,1′-biphenyl]-2-ylmethyl)-6-(dipropylamino)-2,4-dioxo-3,4-dihydro-1,3,5-triazin-1(2H)-yl)benzoic acid;
3-(2,4-dioxo-6-piperidin-1-yl-3-[4-(trifluoromethyl)benzyl]-3,4-dihydro-1,3,5-triazin-1(2H)-yl)benzoic acid;
3-(3-[4-(benzyloxy)benzyl]-2,4-dioxo-6-piperidin-1-yl-3,4-dihydro-1,3,5-triazin-1(2H)-yl)benzoic acid;
3-(3-(3,4-dimethylbenzyl)-2,4-dioxo-6-piperidin-1-yl-3,4-dihydro-1,3,5-triazin-1(2H)-yl)benzoic acid;
3-(3-([1,1′-biphenyl]-2-ylmethyl)-2,4-dioxo-6-piperidin-1-yl-3,4-dihydro-1,3,5-triazin-1(2H)-yl)benzoic acid;
3-(6-[benzyl(ethyl)amino]-3-(4-bromobenzyl)-2,4-dioxo-3,4-dihydro-1,3,5-triazin-1(2H)-yl)benzoic acid;
3-(6-[benzyl(ethyl)amino]-2,4-dioxo-3-[4-(trifluoromethyl)benzyl]-3,4-dihydro-1,3,5-triazin-1(2H)-yl)benzoic acid;
3-(6-[benzyl(ethyl)amino]-3-[4-(benzyloxy)benzyl]-2,4-dioxo-3,4-dihydro-1,3,5-triazin-1(2H)-yl)benzoic acid;
3-(6-[benzyl(ethyl)amino]-3-(3,4-dimethylbenzyl)-2,4-dioxo-3,4-dihydro-1,3,5-triazin-1(2H)-yl)benzoic acid;
3-(6-[benzyl(ethyl)amino]-3-([1,1′-biphenyl]-2-ylmethyl)-2,4-dioxo-3,4-dihydro-1,3,5-triazin-1(2H)-yl)benzoic acid;
3-(3-(4-bromobenzyl)-6-(dipropylamino)-2,4-dioxo-3,4-dihydro-1,3,5-triazin-1(2H)-yl)benzoic acid;
3-(6-(dipropylamino)-2,4-dioxo-3-[4-(trifluoromethyl)benzyl]-3,4-dihydro-1,3,5-triazin-1(2H)-yl)benzoic acid;
3-(3-[4-(benzyloxy)benzyl]-6-(dipropylamino)-2,4-dioxo-3,4-dihydro-1,3,5-triazin-1(2H)-yl)benzoic acid;
3-(3-(3,4-dimethylbenzyl)-6-(dipropylamino)-2,4-dioxo-3,4-dihydro-1,3,5-triazin-1(2H)-yl)benzoic acid;
3-(3-([1,1′-biphenyl]-2-ylmethyl)-6-(dipropylamino)-2,4-dioxo-3,4-dihydro-1,3,5-triazin-1(2H)-yl)benzoic acid;
2-chloro-4-(2,4-dioxo-6-piperidin-1-yl-3-[4-(trifluoromethyl)benzyl]-3,4-dihydro-1,3,5-triazin-1(2H)-yl)benzoic acid;
4-(3-[4-(benzyloxy)benzyl]-2,4-dioxo-6-piperidin-1-yl-3,4-dihydro-1,3,5-triazin-1(2H)-yl)-2-chlorobenzoic acid;
2-chloro-4-(3-(3,4-dimethylbenzyl)-2,4-dioxo-6-piperidin-1-yl-3,4-dihydro-1,3,5-triazin-1(2H)-yl)benzoic acid;
4-(3-([1,1′-biphenyl]-2-ylmethyl)-2,4-dioxo-6-piperidin-1-yl-3,4-dihydro-1,3,5-triazin-1(2H)-yl)-2-chlorobenzoic acid;
4-(6-[benzyl(ethyl)amino]-3-(4-bromobenzyl)-2,4-dioxo-3,4-dihydro-1,3,5-triazin-1(2H)-yl)-2-chlorobenzoic acid;
4-(6-[benzyl(ethyl)amino]-2,4-dioxo-3-[4-(trifluoromethyl)benzyl]-3,4-dihydro-1,3,5-triazin-1(2H)-yl)-2-chlorobenzoic acid;
4-(6-[benzyl(ethyl)amino]-3-[4-(benzyloxy)benzyl]-2,4-dioxo-3,4-dihydro-1,3,5-triazin-1(2H)-yl)-2-chlorobenzoic acid;
4-(6-[benzyl(ethyl)amino]-3-(3,4-dimethylbenzyl)-2,4-dioxo-3,4-dihydro-1,3,5-triazin-1(2H)-yl)-2-chlorobenzoic acid;
4-(6-[benzyl(ethyl)amino]-3-([1,1′-biphenyl]-2-ylmethyl)-2,4-dioxo-3,4-dihydro-1,3,5-triazin-1(2H)-yl)-2-chlorobenzoic acid;
4-(3-(4-bromobenzyl)-6-(dipropylamino)-2,4-dioxo-3,4-dihydro-1,3,5-triazin-1(2H)-yl)-2-chlorobenzoic acid;
2-chloro-4-(6-(dipropylamino)-2,4-dioxo-3-[4-(trifluoromethyl)benzyl]-3,4-dihydro-1,3,5-triazin-1(2H)-yl)benzoic acid;
4-(3-[4-(benzyloxy)benzyl]-6-(dipropylamino)-2,4-dioxo-3,4-dihydro-1,3,5-triazin-1(2H)-yl)-2-chlorobenzoic acid;
2-chloro-4-(3-(3,4-dimethylbenzyl)-6-(dipropylamino)-2,4-dioxo-3,4-dihydro-1,3,5-triazin-1(2H)-yl)benzoic acid;
4-(3-([1,1′-biphenyl]-2-ylmethyl)-6-(dipropylamino)-2,4-dioxo-3,4-dihydro-1,3,5-triazin-1(2H)-yl)-2-chlorobenzoic acid;
2-chloro-5-(2,4-dioxo-6-piperidin-1-yl-3-[4-(trifluoromethyl)benzyl]-3,4-dihydro-1,3,5-triazin-1(2H)-yl)benzoic acid;
5-(3-[4-(benzyloxy)benzyl]-2,4-dioxo-6-piperidin-1-yl-3,4-dihydro-1,3,5-triazin-1(2H)-yl)-2-chlorobenzoic acid;
2-chloro-5-(3-(3,4-dimethylbenzyl)-2,4-dioxo-6-piperidin-1-yl-3,4-dihydro-1,3,5-triazin-1(2H)-yl)benzoic acid;
5-(3-([1,1′-biphenyl]-2-ylmethyl)-2,4-dioxo-6-piperidin-1-yl-3,4-dihydro-1,3,5-triazin-1(2H)-yl)-2-chlorobenzoic acid;
5-(6-[benzyl(ethyl)amino]-3-(4-bromobenzyl)-2,4-dioxo-3,4-dihydro-1,3,5-triazin-1(2H)-yl)-2-chlorobenzoic acid;
5-(6-[benzyl(ethyl)amino]-2,4-dioxo-3-[4-(trifluoromethyl)benzyl]-3,4-dihydro-1,3,5-triazin-1(2H)-yl)-2-chlorobenzoic acid;
5-(6-[benzyl(ethyl)amino]-3-[4-(benzyloxy)benzyl]-2,4-dioxo-3,4-dihydro-1,3,5-triazin-1(2H)-yl)-2-chlorobenzoic acid;
5-(6-[benzyl(ethyl)amino]-3-(3,4-dimethylbenzyl)-2,4-dioxo-3,4-dihydro-1,3,5-triazin-1(2H)-yl)-2-chlorobenzoic acid;
5-(6-[benzyl(ethyl)amino]-3-([1,1′-biphenyl]-2-ylmethyl)-2,4-dioxo-3,4-dihydro-1,3,5-triazin-1(2H)-yl)-2-chlorobenzoic acid;
5-(3-(4-bromobenzyl)-6-(dipropylamino)-2,4-dioxo-3,4-dihydro-1,3,5-triazin-1(2H)-yl)-2-chlorobenzoic acid;
2-chloro-5-(6-(dipropylamino)-2,4-dioxo-3-[4-(trifluoromethyl)benzyl]-3,4-dihydro-1,3,5-triazin-1(2H)-yl)benzoic acid;
5-(3-[4-(benzyloxy)benzyl]-6-(dipropylamino)-2,4-dioxo-3,4-dihydro-1,3,5-triazin-1(2H)-yl)-2-chlorobenzoic acid;
2-chloro-5-(3-(3,4-dimethylbenzyl)-6-(dipropylamino)-2,4-dioxo-3,4-dihydro-1,3,5-triazin-1(2H)-yl)benzoic acid; or
5-(3-([1,1′-biphenyl]-2-ylmethyl)-6-(dipropylamino)-2,4-dioxo-3,4-dihydro-1,3,5-triazin-1(2H)-yl)-2-chlorobenzoic acid; and pharmaceutical salts thereof.

11. A method for the solid phase synthesis of compounds according to the formula (I): 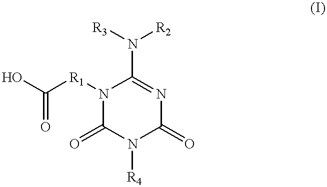
wherein:
R is
an amino acid side chain or phenyl optionally substituted with one or two substituents selected from halogen, straight chain alkyl of 1 to 6 carbon atoms, branched chain alkyl of 2 to 6 carbon atoms, or alkoxy of 1 to 6 carbon atoms;
R² and R³ are independently
hydrogen, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms, phenylalkyl of 7 to 12 carbon atoms, phenyl or acyl; or R² and R³ taken together with the atom to which they are attached, form a 5 or 6 membered heterocyclic ring having one to three heteroatoms selected from O, N and S;
R⁴ is
hydrogen, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms, phenylalkyl of 7 to 12 carbon atoms wherein the phenyl group may be optionally substituted with 1 or 2 substituents selected from
halogen, trifluoromethyl, straight chain or branched alkoxy of 2 to 7 carbon atoms, phenylalkoxy of 7 to 12 carbon atoms, nitro, methylenedioxy, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms or two substituents on adjacent carbons taken together with the phenyl ring form naphthyl optionally substituted with 1 or 2 substituents selected from
halogen, straight chain or branched alkoxy of 2 to 7 carbon atoms, nitro, methylenedioxy, straight chain alkyl of 1 to 6 carbon atoms, branched chain alkyl of 3 to 7 carbon atoms, cycloalkyl of 3 to 7 carbon atoms, straight chain alkenyl of 2 to 6 carbon atoms, branched chain alkenyl of 3 to 9 carbon atoms, straight chain alkynyl of 3 to 9 carbon atoms, branched chain alkynyl of 4 to 9 carbon atoms; and pharmaceutical salts thereof;
comprising the steps of:
a) attaching a Fmoc protected amino acid of the formula 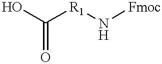
to a solid support to produce a compound of formula (1) 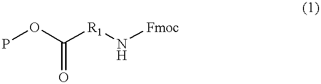
wherein R¹ is as defined above and P is a solid support;
b) deprotecting the compound of formula (1) with piperidine to produce a compound of formula (2) 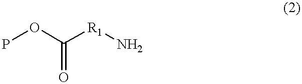
wherein R¹ and P are as defined above;
c) reacting the compound of formula (2) with Fmoc-isothiocyanate to produce a compound of formula (3) 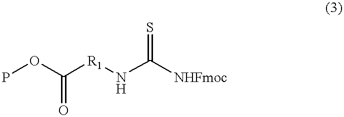
wherein R¹ and P are as defined above;
d) deprotecting the compound of formula (3) with piperidine to produce a compound of formula (4) 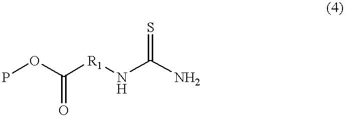
wherein R¹ and P are as defined above;
e) reacting the compound of formula (4) with methyl iodide to produce a compound of formula (5) 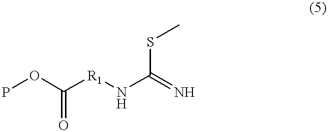
wherein R¹ and P are as defined above;
f) reacting the compound of formula (5) with an amine of formula (6) to produce a compound of formula (7) 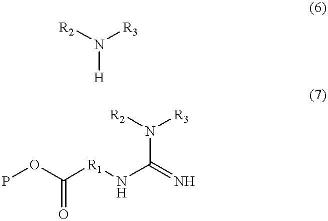
wherein R¹, R², R³ and P are as defined above;
g) reacting compound of formula (7) with chlorocarbonylisocyanate to produce a compound of formula (8) 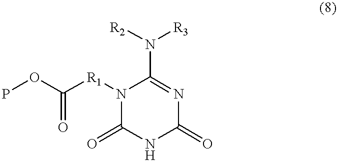
wherein R¹, R², R³ and P are as defined above;
h) reacting said compound of formula (8) with an alcohol under mitsunobu condition to produce a compound of formula (9) 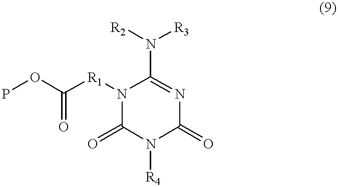
wherein R¹, R², R³ and R⁴ are as defined above;

12. The method of claim 11 further comprising:
i) reacting said compound of formula (9) with a cleaving reagent to produce a compound of formula (I) 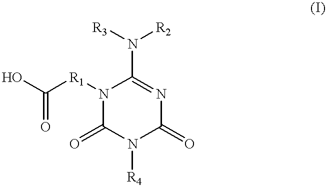
wherein R¹, R², R³ and R⁴ are as defined above.

13. The method according to claim 12 wherein the cleaving reagent is trifluoroacetic acid.

14. The method according to claim 11 wherein the solid support used is polystyrene crosslinked with divinylbenzene and functionalized with a linker such as a hydroxymethylphenoxy group.

15. The method according to claim 11 wherein the solid support used is Wang resin.

16. A pharmaceutical composition comprising a therapeutically effective amount of a compound of claim 1 and a pharmaceutically acceptable carrier.
